(19) United States
(12) Patent Application Publication
(10) Pub. No.: US 2024/0221940 A1
(43) Pub. Date: Jul. 4, 2024

(54) APPARATUS AND METHOD FOR EXPLORING OPTIMIZED TREATMENT PATHWAY THROUGH MODEL-BASED REINFORCEMENT LEARNING BASED ON SIMILAR EPISODE SAMPLING

(71) Applicant: ELECTRONICS AND TELECOMMUNICATIONS RESEARCH INSTITUTE, Daejeon (KR)

(72) Inventors: Do Hyeun KIM, Daejeon (KR); Hwin Dol PARK, Daejeon (KR); Jae Hun CHOI, Daejeon (KR)

(73) Assignee: ELECTRONICS AND TELECOMMUNICATIONS RESEARCH INSTITUTE, Daejeon (KR)

(21) Appl. No.: 18/345,709

(22) Filed: Jun. 30, 2023

(30) Foreign Application Priority Data
Dec. 29, 2022 (KR) 10-2022-0189362

(51) Int. Cl. G16H 50/20; G16H 10/60

(52) U.S. Cl. CPC G16H 50/20; G16H 10/60

(57) ABSTRACT

Disclosed is an apparatus for exploring an optimized treatment pathway of a target patient, which includes an episode sampling module that receives a virtual electronic medical record (EMR) episode, calculates a similarity between a first current state of the target patient, which corresponds to the received virtual EMR episode, and a second current state of a patient, which corresponds to each of a plurality of EMR episodes, extracts an EMR episode, and outputs a pair of the virtual EMR episode and the extracted EMR episode, a state value evaluation module that predicts an expected value of a reward, a treatment method learning module that predicts an optimized treatment method and optimized timing of treatment and provides an external prediction model with the current state of the target patient and the treatment method, and a virtual episode generation module that generates a new virtual EMR episode.

### CROSS-REFERENCE TO RELATED APPLICATIONS

This application claims priority under 35 U.S.C. § 119 to Korean Patent Application No. 10-2022-0189362 filed on Dec. 29, 2022, in the Korean Intellectual Property Office, the disclosures of which are incorporated by reference herein in their entireties.

### BACKGROUND

Embodiments of the present disclosure described herein relate to an artificial intelligence device, and more particularly, relate to an apparatus and a method for exploring an optimized treatment pathway through model-based reinforcement learning based on similar episode sampling.

A medical artificial intelligence technology has been developed in an order of diagnosing whether there is a disease, predicting a patient state, and exploring a treatment method. General medical artificial intelligence is currently applied to a diagnosis of whether there is a disease. Recently, a supervised or unsupervised deep learning technology has been used. Particularly, the medical artificial intelligence technology is frequently used to detect an abnormal part from medical imaging, such as computerized tomography (CT), X-ray, or magnetic resonance imaging (MRI), or analyze a continuous biometric signal. An artificial intelligence technology, such as a convolutional neural network (CNN), a recurrent neural network, or a long short-term memory (LSTM), is mainly used as the medical artificial intelligence technology. Particularly, the medical artificial intelligence technology may be applied to treatment method exploration for finding out which treatment method is most effective for patients. The purpose of exploring the treatment method is to explore a series of treatment pathways for finally improving a patient to the best state. To this end, a reinforcement learning technology may be used.

Due to characteristics of medical environments, the greatest difficulty in applying the reinforcement learning is episode collection. The reinforcement learning requires many episodes to learn an optimized policy. However, because considerable costs are incurred to collect data in which a treatment method is applied to a real patient, it is difficult to ensure a sufficient number of training data. Furthermore, results in which various treatment methods are applied to patients in the same state should be collected to explore an optimized treatment pathway, but there is difficulty in trial and error because there is no patient in substantially the same state.

One of methods for effectively performing the reinforcement learning in a situation where it is difficult to collect sufficient episodes through trial and error from environments is model-based reinforcement learning. The model-based reinforcement learning is to configure a model capable of simulating a real environment, collect virtual episodes through an interaction with a model, and learn a policy of the reinforcement learning based on the collected episodes. When the model finely simulates an environment, the cost of collecting an episode may be saved and a result may be collected and learned even for an action which has not been attempted in the real environment. However, when an inaccurate model is implemented, a result in which a result of an interaction between an action and an environment is distorted occurs. This may hinder learning to an optimized policy.

On the other hand, episode-based reinforcement learning for performing learning only using a collected episode without using a model may also be used. Because the episode-based reinforcement learning is to learn a policy based on data collected from a real environment, there is no distortion capable of occurring due to an inaccurate model, but the direction of learning is unknown for situations except for the actually collected data. Only when there is a reward for a specific action in a specific environment situation, learning is possible to pursue or avoid the action. Because it is unable to accurately estimate a value for the action in a situation where the corresponding data is not collected, there is great difficulty in learning an optimized policy.

When the model-based reinforcement learning is performed for limited medical data, because it is difficult to accurately predict a state change when applying a treatment method to a real patient, the result of collecting episodes using the implemented model may be distorted. On the other hand, when the episode-based reinforcement learning is performed, because a treatment method for patients in the same state is applied only once, it is difficult to explore an optimized treatment pathway because rewards and effectiveness of various treatment methods are unknown. A reward over-estimation phenomenon in which a value for a treatment method which is not attempted is highly estimated may occur. Therefore, there is a need for a research for a method for preventing such reward over-estimation.

### SUMMARY

Embodiments of the present disclosure provide an apparatus and a method for exploring an optimized treatment pathway through model-based reinforcement learning based on similar episode sampling.

According to an embodiment, an apparatus for exploring an optimized treatment pathway of a target patient may include an episode sampling module that receives a virtual electronic medical record (EMR) episode, calculates a similarity between a current state of the target patient, the current state corresponding to the received virtual EMR episode, and a current state of the target patient, the current state corresponding to each of a plurality of EMR episodes, extracts an EMR episode in which the calculated similarity is highest among the plurality of EMR episodes, and outputs a pair of the virtual EMR episode and the extracted EMR episode, a state value evaluation module that predicts an expected value of a reward when performing a specific treatment method for the current state of the target patient, based on the pair of the virtual EMR episode and the extracted EMR episode, a treatment method learning module that predicts an optimized treatment method and optimized timing of treatment capable of maximizing an expected value of a reward of the target patient and provides an external prediction model with the current state of the target patient and the treatment method to obtain a next state of the target patient and a reward, and a virtual episode generation module that generates a new virtual EMR episode based on the treatment method, the timing of treatment, the next state, and the reward.

According to an embodiment, a method for exploring an optimized treatment pathway of a target patient may include calculating a similarity between a current state of the target patient, the current state corresponding to a received virtual electronic medical record (EMR) episode, and a current state of the target patient, the current state corresponding to each of a plurality of EMR episodes, extracting an EMR episode in which the calculated similarity is highest among the plurality of EMR episodes, and outputting a pair of the virtual EMR episode and the extracted EMR episode, predicting an expected value of a reward when performing a specific treatment method for the current state of the target patient, based on the pair of the virtual EMR episode and the extracted EMR episode, predicting an optimized treatment method and an optimized timing of treatment capable of maximizing an expected value of a reward of the target patient, providing an external prediction model with the current state of the target patient and the treatment method to obtain a next state of the target patient and a reward, and generating a new virtual EMR episode based on the treatment method, the timing of treatment, the next state, and the reward.

According to an embodiment, a system for exploring an optimized treatment pathway may include a treatment pathway exploring device that predicts a treatment method for a target patient based on an electronic medical record (EMR) episode and a patient state prediction device that receives a current state of the target patient and the treatment method and outputs a next state of the target patient and a reward. The treatment pathway exploring device may include an episode sampling module that receives a virtual EMR episode, calculates a similarity between a current state of the target patient, the current state corresponding to the received virtual EMR episode, and a current state of the target patient, the current state corresponding to each of a plurality of EMR episodes, extracts an EMR episode in which the calculated similarity is highest among the plurality of EMR episodes, and outputs a pair of the virtual EMR episode and the extracted EMR episode, a state value evaluation module that predicts an expected value of a reward when performing a specific treatment method for the current state of the target patient, based on the pair of the virtual EMR episode and the extracted EMR episode, a treatment method learning module that predicts an optimized treatment method and optimized timing of treatment capable of maximizing an expected value of the reward of the target patient and provides the patient state prediction device with the current state of the target patient and the treatment method to obtain the next state of the target patient and the reward, and a virtual episode generation module that generates a new virtual EMR episode based on the treatment method, the timing of treatment, the next state, and the reward.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating a configuration of a system for exploring an optimized treatment pathway according to an embodiment of the present disclosure;

FIG. 2 is a block diagram illustrating a configuration of a treatment pathway exploring device of FIG. 1;

FIG. 3 conceptually illustrates an operation of a treatment pathway exploring device of FIG. 2;

FIG. 4 conceptually illustrates an operation of an episode sampling module of FIG. 3;

FIG. 5 conceptually illustrates an operation of a state value evaluation module of FIG. 3;

FIG. 6 conceptually illustrates an operation of a treatment method learning module of FIG. 3;

FIG. 7 conceptually illustrates an operation of a virtual episode generation module of FIG. 3; and

FIG. 8 is a flowchart illustrating a method for exploring an optimized treatment pathway according to an embodiment of the present disclosure.

### DETAILED DESCRIPTION

Below, embodiments of the present disclosure will be described in detail and clearly to such an extent that one skilled in the art easily carries out the present disclosure.

In the detailed description, components described with reference to the terms “unit”, “module”, “block”, “-er or -or”, etc. and function blocks illustrated in drawings will be implemented with software, hardware, or a combination thereof. Illustratively, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

A treatment pathway of a disease may be defined as a series of processes for repeating a medical practice (referred to as a “treatment method” in the specification) for examining and improving a patient state until a patient is fully cured or dies. An artificial intelligence technology may recommend a treatment method capable of comparing a next patient state with a current patient state and improving the next patient state the most. However, although the next patient state is significantly improved according to the recommended treatment method, when subsequent treatment worsens the patient state, the recommended treatment method is not an optimized treatment method. Thus, the optimized treatment method is a treatment method which makes a final state of the patient the best, rather than treatment for maximizing the degree of immediate improvement.

To make the final state of the patient the best, there is a need for a consecutive pathway of several treatments capable of being performed stage by stage according to a change in patient state. The present disclosure defines such a consecutive pathway as an optimized treatment pathway for a patient. Exploring the optimized treatment pathway may correspond to exploring sequential decision making for maximizing cumulative reward. In other words, an optimized treatment pathway plan is to iteratively learn a process of correcting a treatment method through trial and error which occurs every time the treatment method is applied to a patient to learn consecutive treatment methods capable of making a final state of the patient the best (i.e., maximizing cumulative reward). To this end, a reinforcement learning method may be used.

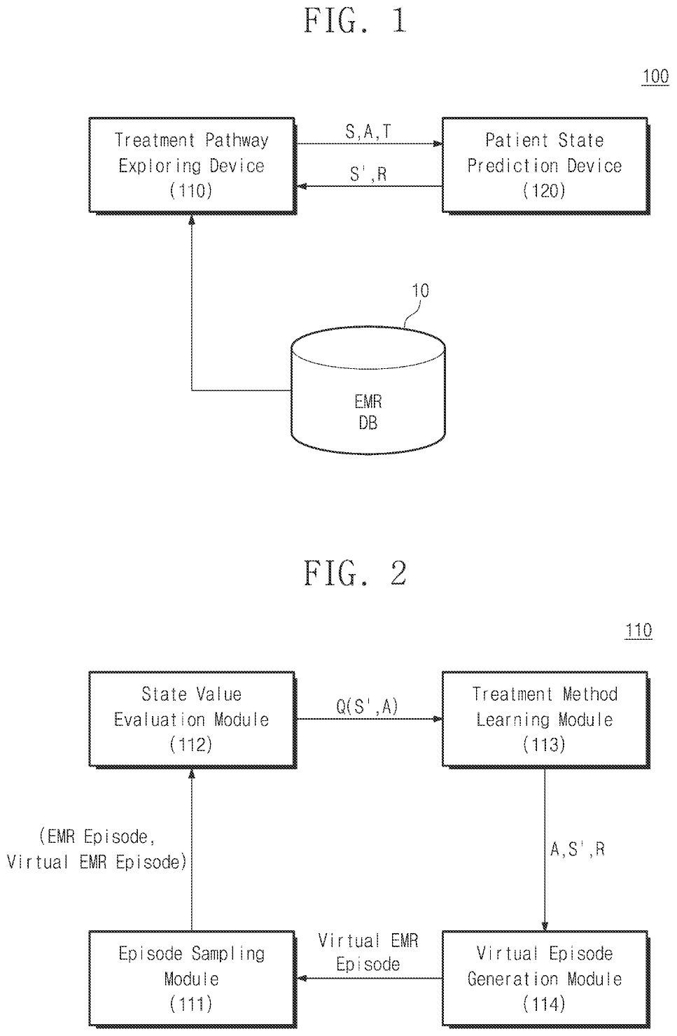

FIG. 1 is a block diagram illustrating a configuration of a system 100 for exploring an optimized treatment pathway according to an embodiment of the present disclosure. Referring to FIG. 1, the system 100 may include a treatment pathway exploring device 110 and a patient state prediction device 120. The treatment pathway exploring device 110 may receive an electronic medical record (EMR) of patients from an EMR database (DB) 10. The EMR may be records about examination and treatment of all patients who visit a medical institution, which are stored in the form of a time series over time. The treatment pathway exploring device 110 may convert such an EMR into a time series episode which is in the form of a patient state S, a treatment method A, a reward R, a time T, and a next state S′ of a patient. The EMR converted into the episode is referred to as an EMR episode.

The treatment pathway exploring device 110 may predict a treatment method corresponding to a patient state based on the EMR episode and may learn the best treatment pathway. The treatment pathway exploring device 110 may deliver the treatment method A and the time T predicted for the current state S of the patient to the patient state prediction device 120. The patient state prediction device 120 may predict the reward R and the next state S′ of the patient based on the received treatment method and time. The treatment pathway exploring device 110 may receive the predicted reward and the predicted next state of the patient from the patient state prediction device 120 and may generate a virtual EMR episode having a form of the patient state S, the predicted treatment method A, the predicted reward R, the predicted time T, and the predicted next state S′ of the patient together with the previously predicted treatment method and time. The treatment pathway exploring device 110 may perform prediction of the above-mentioned treatment method and learning of the treatment pathway using a virtual EMR episode generated through an interaction between the treatment pathway exploring device 110 and the patient state prediction device 120 as well as an EMR episode generated from a real EMR stored in the EMR DB 10. The generated virtual EMR episode may be stored in a temporary memory or a storage device in the treatment pathway exploring device 110.

For example, the treatment pathway exploring device 110 may include an artificial intelligence model for predicting a treatment method and a time, and the patient state prediction device 120 may include an artificial intelligence model (e.g., a time series probability distribution model) for predicting a next state of a patient and a reward. Functions of the treatment pathway exploring device 110 and the patient state prediction device 120 may be implemented using hardware, including combination logic, which executes instructions stored in any type of memory (e.g., a flash memory, such as a NAND flash memory or a low-latency NAND flash memory, a persistent memory (PMEM), such as a cross-grid non-volatile memory, a memory with bulk resistance variation, a phase change memory (PCM), or the like or a combination thereof), sequential logic, one or more timers, counters, registers, state machines, one or more complex programmable logic devices (CPLDs), a field programmable gate array (FPGA), an application specific integrated circuit (ASIC), a central processing unit (CPU), such as complex instruction set computer (CSIC) processors such as x86 processors and/or a reduced instruction set computer (RISC) such as ARM processors, a graphics processing unit (GPU), a neural processing unit (NPU), a tensor processing unit (TPU), an accelerated processing unit (APU), or the like, or a combination thereof, software, or a combination thereof.

Furthermore, operations of the treatment pathway exploring device 110 and the patient state prediction device 120 may be implemented as a program code stored in a non-transitory computer-readable medium. For example, the non-transitory computer-readable medium may include a magnetic medium, an optical medium, or a combination thereof (e.g., a compact disc read-only memory (CD-ROM), a hard drive, a read-only memory (ROM), a flash memory, or the like).

FIG. 2 is a block diagram illustrating a configuration of a treatment pathway exploring device 110 of FIG. 1. Referring to FIG. 2, the treatment pathway exploring device 110 may include an episode sampling module 111, a state value evaluation module 112, a treatment method learning module 113, and a virtual episode generation module 114.

The episode sampling module 111 may receive a virtual EMR episode, may extract a real EMR episode similar to the received virtual EMR episode, and may provide the state value evaluation module 112 with a pair of the real EMR episode and the virtual EMR episode, which are similar to each other. As such, the process of extracting the real EMR episode similar to the virtual EMR episode is referred to as similar episode sampling.

When performing treatment depending on a specific treatment method in a current state of a patient which is a target to explore a treatment pathway, the state value evaluation module 112 may predict an expected value (expressed as Q(S′, A)) of a reward capable of being received until an end time point of the EMR episode. The state value evaluation module 112 may be learned to accurately predict an expected value of a reward. The state value evaluation module 112, the learning of which is completed, may receive any patient state and any treatment method and may predict the expected value of the reward. The state value evaluation module 112 may provide the treatment method learning module 113 with the predicted expected value of the reward.

The treatment method learning module 113 may select a treatment method A capable of maximizing a reward for a current patient state and may predict corresponding timing of treatment. In detail, the treatment method learning module 113 may be learned to select a treatment method capable of maximizing an expected value in the current state of the patient, based on the value of the reward, which is predicted by the state value evaluation module 112. The selected treatment method may be provided to a patient state prediction device 120 of FIG. 1. The treatment method learning module 113 may receive a predicted reward R and a predicted next state S′ of the patient from the patient state prediction device 120 of FIG. 1 and may provide the virtual episode generation module 114 with the reward R and the next state S′ of the patient together with the selected treatment method A.

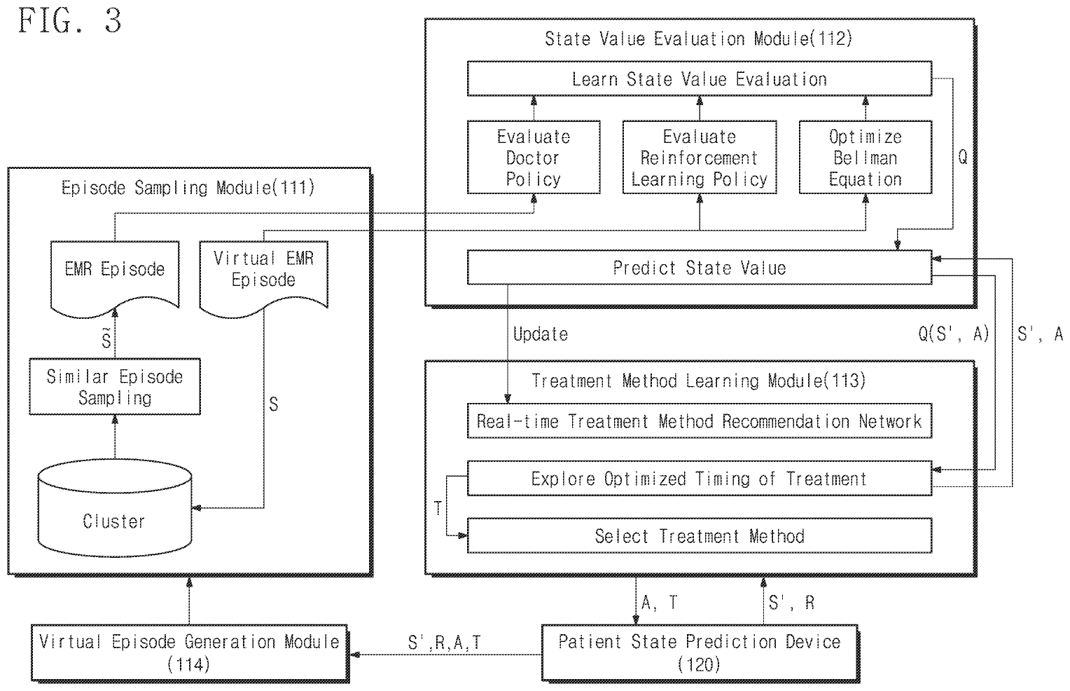

The virtual episode generation module 114 may generate a virtual EMR episode based on the predicted reward, the next state of the patient, and the treatment method and may provide the episode sampling module 111 again with the virtual EMR episode. The generated virtual EMR episode may be used for learning of the state value evaluation module 112 and the treatment method learning module 113, which is described above. Hereinafter, a description will be given of operations of components of the treatment pathway exploring device 110, which are described above, with reference to FIGS. 3 to 7.

FIG. 3 conceptually illustrates an operation of a treatment pathway exploring device 110 of FIG. 2. As described above with reference to FIG. 2, the operation of the treatment pathway exploring device 110 may be performed by an interaction between an episode sampling module 111, a state value evaluation module 112, a treatment method learning module 113, a virtual episode generation module 114, and a patient state prediction device 120.

First of all, the episode sampling module 111 may receive a virtual EMR episode from a temporary memory, which stores the virtual EMR episode, and the virtual episode generation module 114 and may extract a real EMR episode having a state similar to a state S of the received virtual EMR episode (or perform similar episode sampling) from clusters including real EMR episodes having states similar to each other. Thereafter, the episode sampling module 111 may provide the state value evaluation module 112 with a pair of the real EMR episode and the virtual EMR episode, the states of which are similar to each other.

The state value evaluation module 112 may predict an expected value Q(S′, A) of a reward (or predict a state value) when a current state S of a patient and a treatment method A are given, based on the pair of received episodes. At this time, reinforcement learning policy evaluation and optimization of a Bellman equation may be performed for the virtual EMR episode for learning of the state value evaluation module 112. In addition, to minimize an error capable of occurring when an expected value of a reward is predicted with reference to only the virtual EMR episode, doctor policy evaluation (or state value evaluation learning) may be performed for the real EMR episode.

The treatment method learning module 113 may include a network (or a real-time treatment method recommendation network) for recommending a treatment method A corresponding to the state S of the patient, which is given in real time. The real-time treatment method recommendation network may be updated using a function Q learned by the state value evaluation module 112. Furthermore, the treatment method learning module 113 may select optimized timing of treatment T and the optimized treatment method A based on the expected value Q(S′, A) of the reward, may provide the patient state prediction device 120 with the optimized timing of treatment T and the optimized treatment method A, and may provide the virtual episode generation module 114 with a next state S′ of the patient and a reward R, which are predicted by the patient state prediction device 120. The virtual episode generation module 114 may generate a new virtual EMR episode based on the provided values and may provide the episode sampling module 111 with the new virtual EMR episode. The above-mentioned operations may be repeated based on a pair of the new EMR episodes.

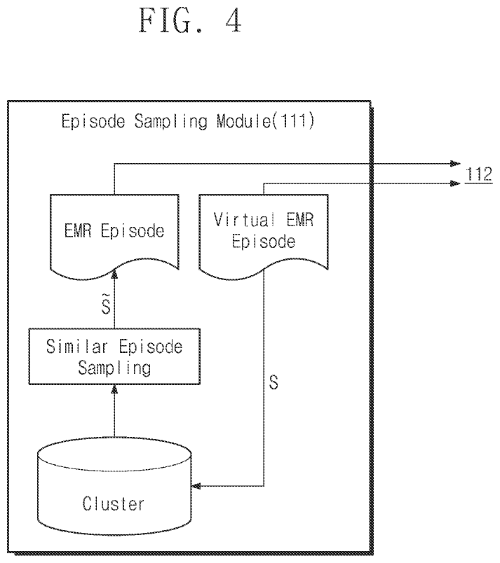

FIG. 4 conceptually illustrates an operation of an episode sampling module 111 of FIG. 3. As described above with reference to FIG. 1, a treatment pathway exploring device 110 may include a temporary memory or a storage device capable of storing a generated virtual EMR episode, and an episode sampling module 111 may receive the virtual EMR episode from the temporary memory or the storage device or may receive a virtual EMR episode generated by a virtual EMR episode generation module 114. Furthermore, the episode sampling module 111 may cluster episodes, each of which has a high similarity of a current state of a patient (or a short geometric distance), among real EMR episodes. For example, the similarity may be calculated as one of a mean square error (MSE) similarity or a cosine similarity and the clustering may be performed through K-mean clustering, but the present disclosure is not limited thereto.

The episode sampling module 111 may extract a real EMR episode having a current state Ŝ similar to a current state S of a patient, which corresponds to a virtual EMR episode input from a cluster (or perform similar episode sampling) and may match the extracted real EMR episode with the received virtual EMR episode. A pair of the real EMR episode and the virtual EMR episode, which are matched with each other, may be provided to a state value evaluation module 112 to evaluate a state value.

Existing reinforcement learning mostly use only any one of a real episode or a virtual episode and does not frequently consider a connection between the real episode and the virtual episode although the real episode and the virtual episode are used together. However, the episode sampling module 111 according to an embodiment of the present disclosure may extract an EMR episode similar to the used virtual EMR episode and may use the extracted EMR episode for learning. As a result, the episode sampling module 111 may reduce an error capable of occurring due to the use of the virtual EMR episode.

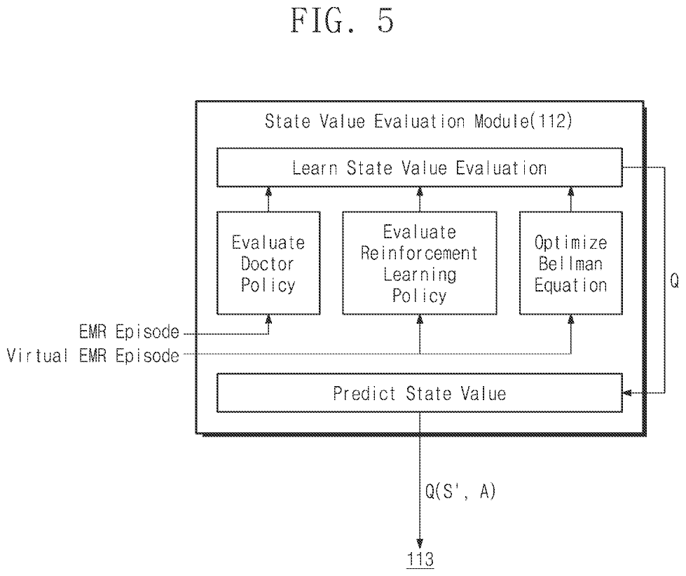

FIG. 5 conceptually illustrates an operation of a state value evaluation module 112 of FIG. 3. The state value evaluation module 112 may receive a pair of a real EMR episode and a virtual EMR episode, which are matched by an episode sampling module 111, and may predict an expected value Q(S′, A) of a future reward when a treatment method A is selected in a given state S of a patient. In detail, the real EMR episode may be used to reflect a real doctor policy to learn a model for state value evaluation, and the virtual EMR episode may be used to learn the model for state value evaluation by means of reinforcement learning policy evaluation and optimization of a Bellman equation.

Herein, learning the model for state value evaluation may be the same as learning a function Q for evaluating a state value, and the function Q may be optimized according to Equation 1 below.

$$\text{?} = arg {min}_{Q} \text{?} α \cdot ( \text{?} [ Q ( s , a ) ] - \text{?} [ Q ( \text{?} , a ) ] ) + \frac{1}{2} \text{?} [ ( Q ( s , a ) - \text{?} ( s , a ) \text{?} ( s , a ) \text{?} ] \text{?} \qquad [ Equation 1 ]$$

$$\text{?} \text{indicates text missing or illegible when filed}$$

Herein, α denotes any weight capable of being adjusted, D denotes the mini-batch including the plurality of EMR episodes, and P denotes the mini-batch including episodes similar to the episodes included in the mini-batch M among the real EMR episodes. S denotes the patient state predicted by the patient state prediction device 120, and \tilde{S} denotes the patient state similar to S, which is the patient state of the real EMR episode.

[symbol] [Q(s, a)] denotes the expected value of the reward calculated through the reinforcement learning Q when attempting to perform the treatment method a for the patient state S, [symbol] [Q(\tilde{s}, a)] and denotes the expected value of the reward calculated through the reinforcement learning Q when attempting to perform the treatment method a for the real patient state \tilde{S} similar to the patient state S. For example, the reward may correspond to a degree to which the patient has improved. μ and π indicate the learned doctor policy and the real doctor policy, respectively. The state value evaluation module 112 may learn the function Q such that the expected value [symbol] [Q(s, a)] of the function Q using the virtual EMR episode is induced to be low and the expected value [symbol] _{\tilde{s}˜P,a˜\tilde{π}}_{_{β}}_{(a|} [symbol] ₎[Q(\tilde{s}, a)] of the function Q using the real EMR episode is induced to be high. As such, over-estimation of the reward using the virtual EMR episode may be prevented, and the real doctor policy may be reflected.

In other words, [symbol] [Q(s, a)]− [symbol] _{s·p,a˜\tilde{π}}_{_{β}}_{(a|\tilde{s})}[Q(\tilde{s}, a)] indicates a difference between an expected value of a reward corresponding to the patient state S and the treatment method a through reinforcement learning Q and an expected value of a reward corresponding to a similar patient state \tilde{S} and the treatment method a. When the reinforcement learning Q plans the treatment method a for the patient state S and when a real medical institution also plans the treatment method a for the patient state \tilde{S}, because the difference between the two expected values is able to be minimized, parameters of a model for state value evaluation may be updated such that the reinforcement learning Q is reinforced in the direction of selection. On the other hand, when the reinforcement learning Q plans the treatment method a in the state S, but when the real medical institution does not perform the treatment method a for the patient state \tilde{S}, the difference between the two expected values may increase and the parameters of the model for state value evaluation may be updated such that the reinforcement learning Q is reinforced in the direction of being not selected. [symbol] ^{π}(s, a)\hat{Q}^{k}(s, a) corresponds to a general Bellman equation.

Thus, according to Equation 1 above, the reinforcement learning Q to minimize the difference between the expected value of the reward when planning the treatment method for the virtual patient state and the expected value of the reward when the medical institution plans the treatment method for the real patient state may be selected (i.e., Q capable of minimizing both of two differences may be selected, argmin_{Q}), and an optimization method of general reinforcement learning may be used together. As such, according to an embodiment of the present disclosure, reinforcement learning in which model-based reinforcement learning and episode-based reinforcement learning through similar episode sampling are combined with each other may be applied to medical data. The learned function Q and the Q(S′, A) value calculated using the learned Q may be provided to the treatment method learning module 113 to be used to update a real-time treatment method recommendation network and explore the optimized timing of treatment.

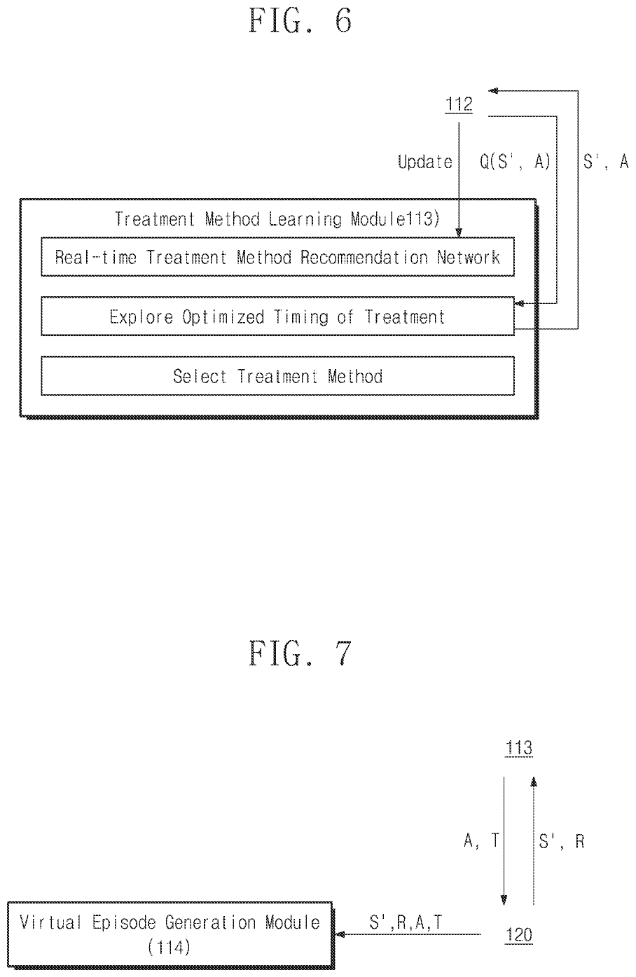

FIG. 6 conceptually illustrates an operation of a treatment method learning module 113 of FIG. 3. As described above, the treatment method learning module 113 may include a real-time treatment method recommendation network (e.g., implemented with a neural network such as a convolutional neural network (CNN) or a recurrent neural network (RNN)) for recommending a treatment method suitable for a patient state in real time. Several parameters of the real-time treatment method recommendation network may be continuously updated based on a function Q learned from a state value evaluation module 112. For example, the parameters of the real-time treatment method recommendation network may be updated to minimize a [symbol] _{π}[Q^{π}(s, a)Λ₀lnπ₀(a|s)] value. Herein, Λ₀lnπ₀(a|s) corresponds to a differential value of a policy π. As such, the real-time treatment method recommendation network may be updated to select a treatment method A for maximizing a Q(S, A) value.

Furthermore, the treatment method learning module 113 may select and predict optimized timing of treatment and an optimized treatment method. First of all, the treatment method A for a given state S of a patient may be predicted through the above-mentioned network. To explore the optimized timing of treatment, the treatment method learning module 113 may input the patient state S and the predicted treatment method A to a patient state prediction device 120 at uniform time intervals T₁˜Tₙ (i.e., (S, A, T₁), (S, A, T₂), . . . , and (S, A, Tₙ)). The patient state prediction device 120 may return a next state S′ of the patient, which corresponds to each time point (i.e., (S′, A, T₁), (S′, A, T₂), . . . , or (S′, A, Tₙ)). The returned n patient states S′ and treatment methods A may be delivered to the state value evaluation module 112. The state value evaluation module 112 may calculate n Q(S′, A) and may obtain a value Q(S′, A) with a maximum reward. At this time, a time point with a maximum reward may be output as optimized timing of treatment T. When the value Q(S′, A) with the maximum reward is obtained, the treatment method learning module 113 may update the parameters of the above-mentioned real-time treatment method recommendation network again using it. An optimized treatment method A may be output through the treatment method recommendation network which is updated again. As such, according to an embodiment of the present disclosure, reinforcement learning considering a time interval may be possible.

FIG. 7 conceptually illustrates an operation of a virtual episode generation module 114 of FIG. 3. The virtual episode generation module 114 may generate a virtual EMR episode including a next state S′ of a patient and a reward R, corresponding to optimized timing of treatment T, among optimized timing of treatment T and a treatment method A received from a treatment method learning module 113 and values predicted by a patient state prediction device 120. The generated virtual EMR episode may be stored in a temporary memory or a storage device in a treatment pathway exploring device 110 and may be used as an input of an episode sampling module 111 in the future.

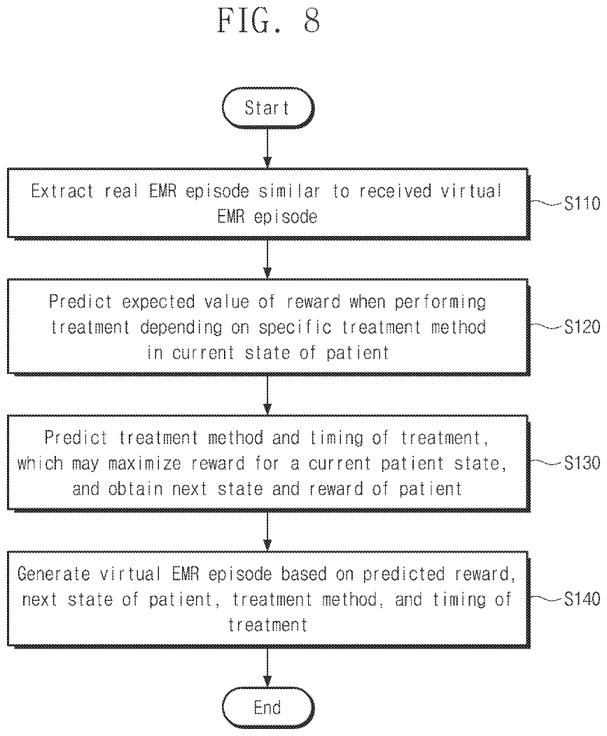

FIG. 8 is a flowchart illustrating a method for exploring an optimized treatment pathway according to an embodiment of the present disclosure. Hereinafter, a description will be given with reference to FIG. 2 together with FIG. 8.

In operation S110, an episode sampling module 111 may extract a real EMR episode similar to a received virtual EMR episode to match the real EMR episode with the virtual EMR episode and may provide a state value evaluation module 112 with a pair of the real EMR episode and the virtual EMR episode. In operation S120, the state value evaluation module 112 may predict an expected value of a reward when performing treatment depending on a specific treatment method in a current state of a patient and may learn a function Q for obtaining the expected value of the reward.

In operation S130, a treatment method learning module 113 may predict a treatment method and optimized timing of treatment capable of maximizing a reward for the current state of the reward through an interaction with a patient state prediction device 120. In detail, the treatment method learning module 113 may provide the patient state prediction device 120 with the current state of the patient and the treatment method to obtain the predicted next state of the patient and the predicted reward. In operation S140, a virtual episode generation module 114 may generate a virtual EMR episode based on the predicted reward, the next state of the patient, the treatment method, and the optimized timing of treatment. The generated virtual EMR episode may be stored in a temporary memory or a storage device in a treatment pathway exploring device 110 and may be used as an input of the episode sampling module 111.

According to an embodiment of the present disclosure, the model-based reinforcement learning and the episode-based reinforcement learning may be applied together to medical data. When the model-based reinforcement learning and the episode-based reinforcement learning are used, it may be more easy to learn a policy with reference to a real doctor's treatment method than virtual episode-based reinforcement learning.

In addition, according to an embodiment of the present disclosure, timing of treatment and a treatment method with maximum treatment utility may be explored for medical data having an irregular time interval. A time interval may be considered in reinforcement learning.

The above-mentioned contents are detailed embodiments for executing the present disclosure. Embodiments in which a design is changed simply or which are easily changed may be included in the inventive concept as well as an embodiment described above. In addition, technologies that are easily changed and implemented by using the above embodiments may be included in the present disclosure. Therefore, the spirit and scope of the present disclosure is defined not by the above-described embodiments, but by those that are identical or equivalent to the claims of the present disclosure as well as the appended claims, which will be described below.

What is claimed is:

1. An apparatus for exploring an optimized treatment pathway of a target patient, the apparatus comprising:
an episode sampling module configured to receive a virtual electronic medical record (EMR) episode, calculate a similarity between a first current state of the target patient, the target patient corresponding to the received virtual EMR episode, and a second current state of a patient, the patient corresponding to each of a plurality of EMR episodes, extract an EMR episode in which the calculated similarity is highest among the plurality of EMR episodes, and output a pair of the virtual EMR episode and the extracted EMR episode;
a state value evaluation module configured to predict an expected value of a reward when performing a specific treatment method for the current state of the target patient, based on the pair of the virtual EMR episode and the extracted EMR episode;
a treatment method learning module configured to predict an optimized treatment method and optimized timing of treatment capable of maximizing the expected value of a reward of the target patient and provide an external prediction model with the current state of the target patient and the treatment method to obtain a next state of the target patient and a reward; and
a virtual episode generation module configured to generate a new virtual EMR episode based on the treatment method, the timing of treatment, the next state, and the reward.

2. The apparatus of claim 1, wherein the episode sampling module calculates the similarity using any one of a mean square error (MSE) similarity or a cosine similarity.

3. The apparatus of claim 1, wherein the state value evaluation module learns a function Q for predicting the expected value of the reward depending on Equation 1 below,
$$\text{?} = arg {min}_{Q} \text{?} α \cdot ( \text{?} [ Q ( s , a ) ] - \text{?} [ Q ( \text{?} , a ) ] ) + \frac{1}{2} \text{?} [ ( Q ( s , a ) - \text{?} ( s , a ) \text{?} ( s , a ) \text{?} ] \text{?} \qquad ( Equation 1 )$$
$$\text{?} \text{indicates text missing or illegible when filed}$$
where α denotes any weight capable of being adjusted, D denotes a mini-batch including a plurality of virtual EMR episodes, P denotes a mini-batch including the plurality of EMR episodes, S denotes the first current state of the target patient, the target patient corresponding to the received virtual EMR episode, Ŝ denotes the second current state of the patient, the second current state being similar to S and the patient corresponding to the extracted EMR episode, a denotes the treatment method, μ and π denote a virtual policy and a real doctor policy, respectively, and [symbol] ^{π}(s, a)\hat{Q}^{k}(s, a) denotes a Bellman equation.

4. The apparatus of claim 1, wherein the treatment method learning module includes a real-time treatment method recommendation network configured to receive the current state of the target patient and output the treatment method.

5. The apparatus of claim 4, wherein the treatment method learning module provides a patient state prediction device with the current state and the treatment method with respect to a plurality of time points to obtain a next state of the target patient, the next state corresponding to each of the plurality of time points, predicts a time point when there is a maximum value of the expected value of the reward calculated based on each of the obtained next states and the treatment method as the optimized timing of treatment, and updates the real-time treatment method recommendation network based on the maximum value of the expected value of the reward and predicts a treatment method being output by inputting the current state of the target patient to the updated network as the optimized treatment method.

6. The apparatus of claim 4, wherein the real-time treatment method recommendation network is updated to select a treatment method for maximizing the expected value of the reward among a plurality of treatment methods.

7. A method for exploring an optimized treatment pathway of a target patient, the method comprising:
calculating a similarity between a first current state of the target patient, the target patient corresponding to a received virtual electronic medical record (EMR) episode, and a second current state of a patient, the patient corresponding to each of a plurality of EMR episodes, extracting an EMR episode in which the calculated similarity is highest among the plurality of EMR episodes, and outputting a pair of the virtual EMR episode and the extracted EMR episode;
predicting an expected value of a reward when performing a specific treatment method for the current state of the target patient, based on the pair of the virtual EMR episode and the extracted EMR episode;
predicting an optimized treatment method and an optimized timing of treatment capable of maximizing the expected value of a reward of the target patient;
providing an external prediction model with the current state of the target patient and the treatment method to obtain a next state of the target patient and a reward; and
generating a new virtual EMR episode based on the treatment method, the timing of treatment, the next state, and the reward.

8. The method of claim 7, wherein the outputting of the pair of the virtual EMR episode and the extracted EMR episode includes:
calculating the similarity using any one of an MSE similarity or a cosine similarity.

9. The method of claim 7, wherein the predicting of the expected value of the reward includes:
learning a function Q for predicting the expected value of the reward depending on Equation 1 below,
$$\text{?} = arg {min}_{Q} \text{?} α \cdot ( \text{?} [ Q ( s , a ) ] - \text{?} [ Q ( \text{?} , a ) ] ) + \frac{1}{2} \text{?} [ ( Q ( s , a ) - \text{?} ( s , a ) \text{?} ( s , a ) \text{?} ] \text{?} \qquad ( Equation 1 )$$
$$\text{?} \text{indicates text missing or illegible when filed}$$
where α denotes any weight capable of being adjusted, D denotes a mini-batch including a plurality of virtual EMR episodes, P denotes a mini-batch including the plurality of EMR episodes, S denotes the first current state of the target patient, the target patient corresponding to the received virtual EMR episode, Ŝ denotes the second current state of the patient, the second current state being similar to S and the patient corresponding to the extracted EMR episode, a denotes the treatment method, μ and π denote a virtual policy and a real doctor policy, respectively, and [symbol] ^{π}(s, a)\hat{Q}^{k}(s, a) denotes a Bellman equation.

10. The method of claim 7, wherein the predicting of the optimized treatment method and the optimized timing of treatment includes:
inputting the current state of the target patient to a real-time treatment method recommendation network and outputting the treatment method;
providing a patient state prediction device with the current state and the treatment method with respect to a plurality of time points to obtain a next state of the target patient, the next state corresponding to each of the plurality of time points;
predicting a time point when there is a maximum value of the expected value of the reward calculated based on each of the obtained next states and the treatment method as the optimized timing of treatment;
updating the real-time treatment method recommendation network based on the maximum value of the expected value of the reward; and
predicting a treatment method being output by inputting the current state of the target patient to the updated network as the optimized treatment method.

11. A system for exploring an optimized treatment pathway, the system comprising:
a treatment pathway exploring device configured to predict a treatment method for a target patient based on an electronic medical record (EMR) episode; and
a patient state prediction device configured to receive a current state of the target patient and the treatment method and output a next state of the target patient and a reward,
wherein the treatment pathway exploring device includes:
an episode sampling module configured to receive a virtual EMR episode, calculate a similarity between the current state of the target patient, the target patient corresponding to the received virtual EMR episode, and the current state of a patient, the patient corresponding to each of a plurality of EMR episodes, extract an EMR episode in which the calculated similarity is highest among the plurality of EMR episodes, and output a pair of the virtual EMR episode and the extracted EMR episode;
a state value evaluation module configured to predict an expected value of a reward when performing a specific treatment method for the current state of the target patient, based on the pair of the virtual EMR episode and the extracted EMR episode;
a treatment method learning module configured to predict an optimized treatment method and optimized timing of treatment capable of maximizing the expected value of the reward of the target patient and provide the patient state prediction device with the current state of the target patient and the treatment method to obtain the next state of the target patient and the reward; and
a virtual episode generation module configured to generate a new virtual EMR episode based on the treatment method, the timing of treatment, the next state, and the reward.
